(19) United States
(12) Patent Application Publication
(10) Pub. No.: US 2005/0055103 A1
(43) Pub. Date: Mar. 10, 2005

(54) Femoral hip prosthesis part, a set of such femoral parts and the production method thereof

(76) Inventors: Francois Badatcheff, Angers (FR); Jean-Pierre Besse, Louviers (FR); Jean-Claude Moretton, Chantraine (FR); Patrice Rochereau, Duran (FR); Peter Thumler, Dusselforf (DE); Dominique Poitout, Marseille (FR); Nathalie Laurent-Renaud, Lyon (FR)

Correspondence Address: YOUNG & THOMPSON, 745 SOUTH 23RD STREET, 2ND FLOOR, ARLINGTON, VA, 22202, US

(21) Appl. No.: 10/491,132

(22) PCT Filed: Sep. 26, 2002
(86) PCT No.: PCT/FR02/03292

(30) Foreign Application Priority Data
Sep. 28, 2001 (FR) 01 12552

(51) Int. Cl. A61F 2/32

(52) U.S. Cl. 623/22.42; 623/23.35; 623/911

(57) ABSTRACT

The invention relates to a femoral prosthesis part, the metaphyseal part of which comprises a series of geometric sections (SA to SG) which are disposed in parallel to the transverse plane at equidistant levels. The aforementioned geometric sections are provided with a quadrilateral shape having strongly rounded summits. The two long sides of said sections converge in the medial direction and each section lies inside a geometric quadrangle which is located in the plane. The four sides of said quadrangle intercept, respectively, four geometric lines (m, l, a, p) which are obtained by projecting the profiles of the internal surface of the cortical bone. The above-mentioned geometric sections pivot in relation to one another in internal rotation from the bottom upwards in order to obtain the desired helitorsion angle.

The present invention relates to an improvement to hip femoral parts of the type comprising a shank, a metaphyseal portion, and a head possessing or suitable for receiving a femoral head prosthesis, said femoral prosthesis part being for inserting and securing in the femur after resection of the neck of the femur. The invention also relates to a set of such parts, so as to provide a plurality of prostheses of different sizes to match patients of different sizes, and it also relates to a method of manufacturing the femoral prosthesis part of the invention and the set combining a plurality of such parts.

In known manner, a femoral prosthesis part comprises a prosthesis shank for inserting into the diaphyseal medullary canal of the femur, followed by a prosthesis metaphyseal portion for fixing in the metaphysis of the femur beneath the line where the neck was subjected to resection, which is conventionally performed with an angle of 30° relative to the axis of the femur, and a head forming a ball, or designed to receive a ball, to replace the femur head proper which has been resected, said prosthesis head usually being in the form of a frustoconical extension for receiving a ball that constitutes a prosthetic head, the axis of the head being suitably inclined and presenting an anteversion of 8° to 20°, and conventionally of 15° relative to the anatomical frontal plane.

In an attempt to match as closely as possibly the anatomical shape of the metaphyseal receptacle constituted by the metaphyseal cortex below the line of resection, it is known to impart helitorsion to the metaphyseal portion of the prosthesis femoral part, such helitorsion providing a better match to the anatomy of the upper portion of the femur and also being intended to reduce, where possible, the loss of bone material that results from the surgeon using a rasp prior to putting the prosthesis per se into place. Such helitorsion, which is conventionally in the range 5° to 10°, or more, in the direction of internal rotation, is generally obtained either by progressive turning of successive horizontal section planes of the metaphyseal portion of the prosthesis about a geometrical axis, or else empirically, e.g. by molding or other methods.

Such known prostheses do not give full satisfaction, in particular because they do not fit closely against the internal surface of the bone cortex, which can lead to points of stress concentration with all the consequences that arise therefrom, both from the point of view of giving pain and from the point of view of prosthesis retention. Ideally, a prosthesis stem, i.e. the femoral part of a hip prosthesis, should make only light contact with the various points of the inside surface of the metaphyseal cortex, while nevertheless being held appropriately because of the large area of engagement. Nevertheless, this ideal situation is particularly difficult to achieve, in particular because of the fact that in a given population of normal femoral anatomy, i.e. close to the mean, there nevertheless exist differences presenting an effect that becomes amplified once the outside shape of the metaphyseal surface of the prosthesis portion is not properly designed.

The present invention seeks to remedy those drawbacks and to provide a femoral part for a hip prosthesis which minimizes or eliminates the risks of poor contact and of exaggerated stresses being created in the femoral metaphysis.

Another object of the invention is to provide such a prosthesis which is very well matched to the femoral metaphyseal anatomy of most patients.

Another object of the invention is to provide such a prosthesis which enables such support to be obtained using a rasp while minimizing the loss of bone matter prior to implantation.

Another object of the invention is to provide a set of prostheses of the invention suitable for matching patients of different sizes, it being understood that the transformation from one prosthesis size to another is not merely a question of scale, since different sizes are not geometrically similar.

Another object of the invention is to provide a method of manufacturing such prostheses and such a set of prostheses, suitable for being implemented in simple manner.

The invention provides a prosthesis femoral part of the type comprising a prosthesis shank, a prosthesis metaphyseal portion for forming a bearing zone in the metaphysis of the femur, and a head having a suitable anteversion angle, said metaphyseal portion being shaped to present helitorsion, the prosthesis being characterized in that the metaphyseal portion presents a succession of geometrical sections at equidistant levels parallel to a plane that is transverse relative to the general direction of the prosthesis, which sections are in the form of quadrilaterals with greatly rounded corners with two major sides that converge in the medial direction, each section being inscribed in, and tangential on all four sides to, a geometrical quadrilateral situated in the plane of said section at the level in question, which quadrilateral intercepts via its four sides the four respective geometrical lines obtained by projecting on two inclined planes, preferably two perpendicular planes such as the sagittal plane and the frontal plane, the profiles, preferably the lateral, medial, anterior and posterior profiles, of the inside surface of the bone cortex of the femur, said sections being turned relative to one another in an inwardly-turning angular direction on passing upwards from one equidistant plane to a consecutive equidistant plane, said turns together leading to the desired helitorsion angle.

Preferably, the helitorsion angle is of the order of 6° to 12°, and more preferably of the order of 8° to 10°.

In a particularly preferred embodiment, the helitorsion is no linearly progressive, and preferably extends in such a manner that if seven consecutive equidistant geometrical section planes are considered starting from the bottom metaphyseal section and going to the top metaphyseal section, which forms the proximal metaphyseal section, the various helitorsion angles from one section to the next starting from the bottom are, for a total helitorsion of 8°: 30′, 30′, 1°, 1°30′, 2°, 2°30′, each of said values possibly varying by ±30′. The individual values for other total helitorsion values can be deduced in proportional manner.

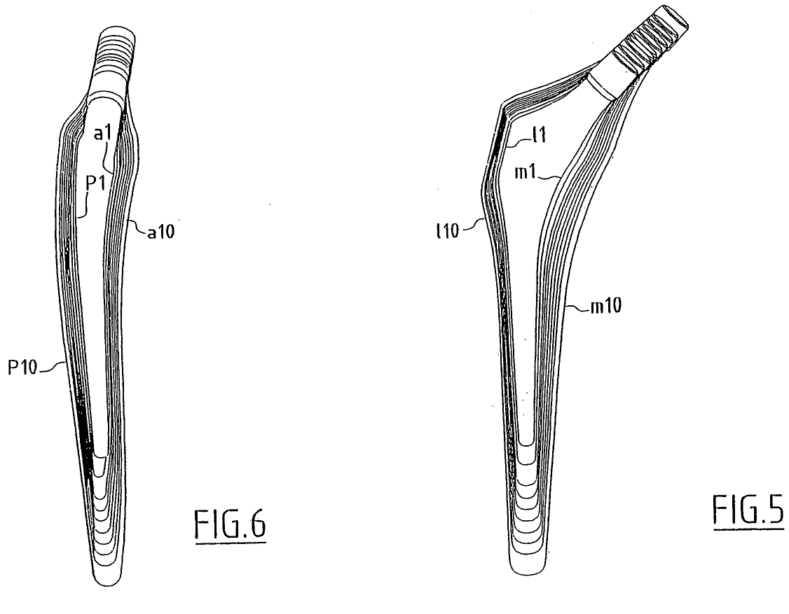

In a particularly preferred embodiment, the four above-mentioned profiles are those which appear in accompanying FIGS. 5 and 6, depending on prosthesis size.

A highly complex shape is thus obtained for the metaphyseal portion, with this shape nevertheless being perfectly reproducible, and being suitable for fitting closely with maximum matching to the inside surface of the femoral cortex of a patient having normal femoral anatomy and having the corresponding size. Naturally, the number of different sizes may be greater or smaller than shown in FIG. 1, and the corresponding profiles may then be extrapolated appropriately.

In a preferred embodiment of the invention, the convergence angle between the two major sides of each geometrical section is of the order of 5° to 10°.

This angle may advantageously vary with the size of the prosthesis, for example being increased for larger prostheses.

The major sides of the rounded quadrilateral section may present respective central rectilinear portions, but in a variant, provision may also be made for the major sides to be curved with a large radius of curvature.

In a suitable embodiment of the invention, these large radii of curvature may remain substantially constant for all of the sections. In an advantageous embodiment, the diaphyseal portion of the prosthesis may be extended in continuity with the metaphyseal portion, tapering and following a single central line of curvature, already known per se in femoral anatomy, with the bottom end of the intra-diaphyseal stem preferably being chamfered or inclined in its posterior portion, said prosthesis thus extending without double curvature, i.e. without any inversion of curvature.

In its metaphyseal portion and/or its diaphyseal portion, the prosthesis may include grooves to facilitate bonding and osteo-integration. These grooves may advantageously be horizontal in the upper metaphyseal portion and longitudinal in the lower part of the metaphyseal portion and in the following prosthesis shank. Advantageously, the upper horizontal groove may define a reference point at its lateral end, which the surgeon can use when putting the prosthesis in place, so as to cause the reference point to coincide with the bottom edge of the resection plane of the neck of the femur.

In a particularly preferred embodiment, the longitudinal groove(s) which may be connected in curved manner with the lower horizontal groove(s), and may follow the anatomical shape of the prosthesis shank in the lower metaphyseal portion and in the diaphyseal portion so that during insertion of the prosthesis into the femur shaft, the groove producing a guidance effect without forcing against the bone material.

The invention also provides a set of hip prosthesis femoral parts, each stem having the above-defined characteristics and differing from the other stems in the set firstly by its size and secondly by its cross-section line being inscribed in the set of profile lines corresponding to its size, as defined in FIGS. 5 and 6, for example.

It should be understood that FIGS. 5 and 6 show a set of ten different sizes of prosthesis. For a set having some other number of sizes of prosthesis, the curves of the profiles in these figures should naturally be slightly modified by interpolation.

The invention also provides a method of manufacturing a prosthesis of the invention, characterized in that for a given size of prosthesis, four projection profiles are determined by projecting the corresponding inside cortex surfaces, e.g. by X-ray or by scanner, preferably onto two perpendicular planes, in particular the sagittal and frontal planes giving lateral, medial, anterior, and posterior profiles, in that a quadrilateral, in particular a rectangle, is determined in a plane which is determined as one of a succession of equidistant parallel cross-section planes the spaced apart along the metaphyseal portion of the prosthesis, with the bottom plane forming the distal metaphyseal section, in that in each of these planes, quadrilaterals are defined of sides that remain respectively parallel, said quadrilaterals intersecting the various profile curves with their different sides in the plane at the level under consideration, and in that there is drawn in each of the quadrilaterals a section of the metaphyseal portion which is tangential to the sides of the quadrilateral in question, having major sides that are inclined relative to the major sides of the section immediately above by a given helitorsion angle, the sum of said helitorsion angles from the proximal section to the distal section determining the total helitorsion angle, in that the data set determined in this way is stored, and in that a femur head manufacturing device is controlled thereby, said device being of any type, e.g. operating by molding, machining, or forging.

The calculations for determining the data, data storage, and the use of the data for controlling the manufacturing device can be implemented in conventional computer means, for example computer-assisted design (CAD) means.

The invention also provides a method of manufacturing a set of such prostheses, characterized in that the above-mentioned method is implemented for each individual prosthesis, all of the prostheses in the set being designed by said method as a function of the size defined by the projection profiles of internal cortex surfaces shown in FIG. 1.

The prosthesis may be made out of any suitable material, for example out of TA6V titanium. It may be coated completely or in part in a coating that promotes osteo-integration, for example a hydroxyapatite coating, and it may optionally present a special surface state, as is known in this field.

In a preferred implementation of the method of manufacture of the invention, angular variation, e.g. for a total helitorsion angle of 8°, is such that when seven equidistant planes are taken into consideration going from the proximal plane to the distal plane, the angular variation is greater towards the proximal plane than towards the distal plane, with this variation preferably being by decreasing increments going from the proximal plane towards the distal plane, with an increment lying in the range 15′ to 3°, for example.

A helitorsion angle of 8° relative to the reference plane determined by the condyles of the femur is preferred. If a different angle is selected, preferably lying in the range 6° to 12°, e.g. 10°, the variations from one plane to the next are preferably extrapolated.

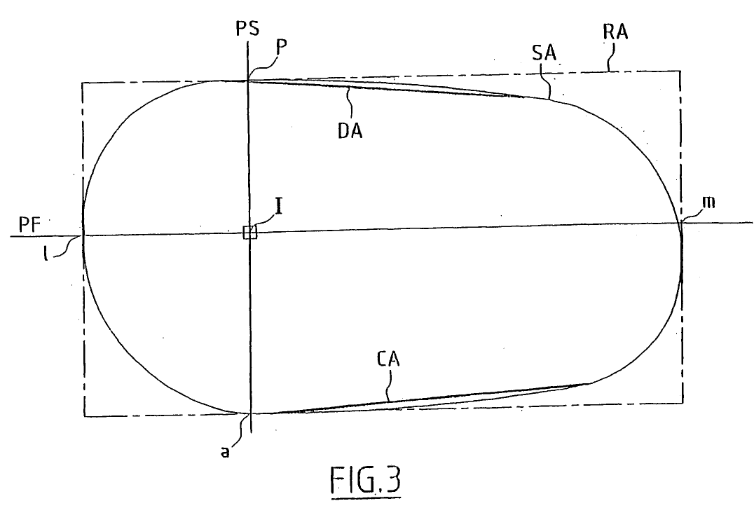

A geometrical axis is advantageously defined forming a point in the proximal plane, said geometrical axis being, for example, substantially a mean diaphyseal axis. By way of example, the sides of the rectangle of the proximal section can be identified, for example, by values Xₘₗ and Yₐₚ, as shown in FIG. 3, with the proportions of the major side and of the minor side of the rectangle being formed relative to the distance ml, i.e. the distance between the points m and l in the proximal plane, thus making it easier to determine the proximal quadrilateral for each prosthesis portion, for example:
Xₘₗ=45% ml
Yₐₚ=28% ml.

Other advantages and characteristics of the invention will appear on reading the following description given by way of non-limiting example and made with reference to the accompanying drawings, in which:

FIG. 1 shows, by way of example, the lateral and medial profiles of the inside surface of the mean diaphyseal cortex for a particular size of prosthesis, as projected onto a frontal plane.

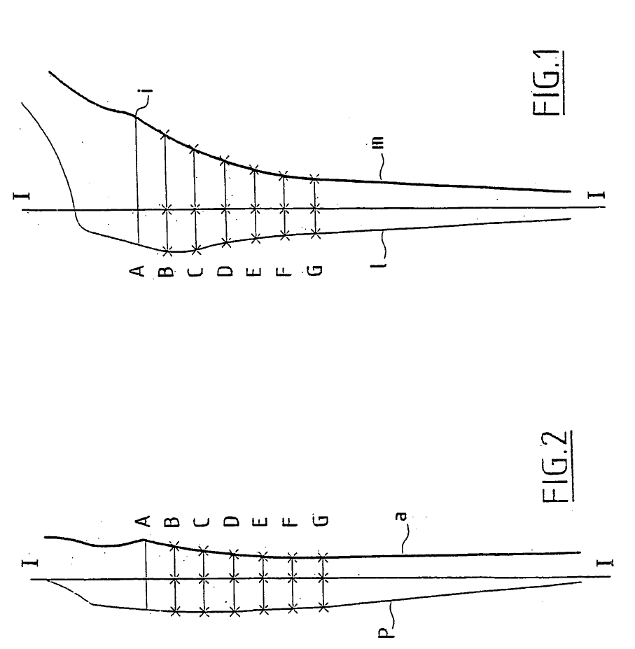

FIG. 2 shows the anterior and posterior director lines projected onto a sagittal plane for the same size of prosthesis.

FIG. 3 shows an example of a cross-section of the prosthesis in a horizontal plane inscribed in a quadrilateral defined at the level of said horizontal section plane.

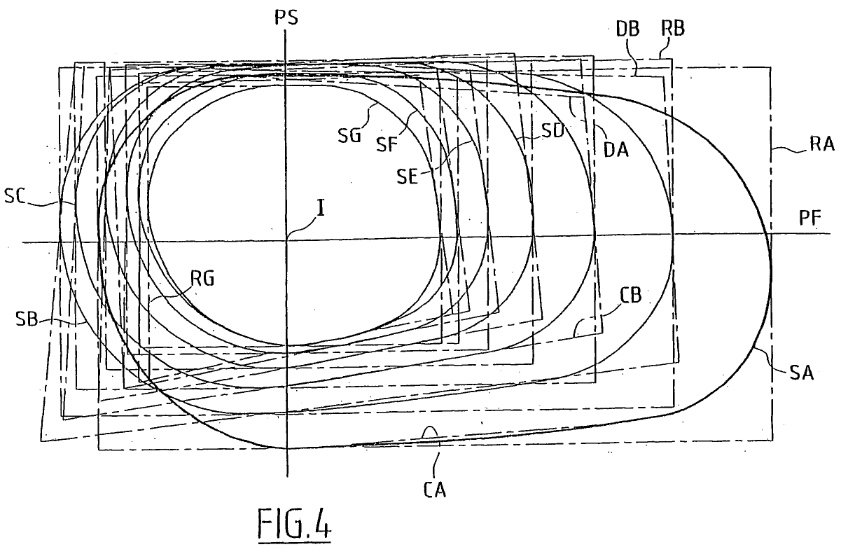

FIG. 4 is a plan view of said horizontal section in the seven section planes corresponding to FIGS. 1 and 2.

FIG. 5 shows the outlines projected onto the frontal plane of a set of ten prostheses of different sizes of the invention, said sizes being known as T1 to T7.

FIG. 6 shows the outlines of the same set when projected onto the sagittal plane.

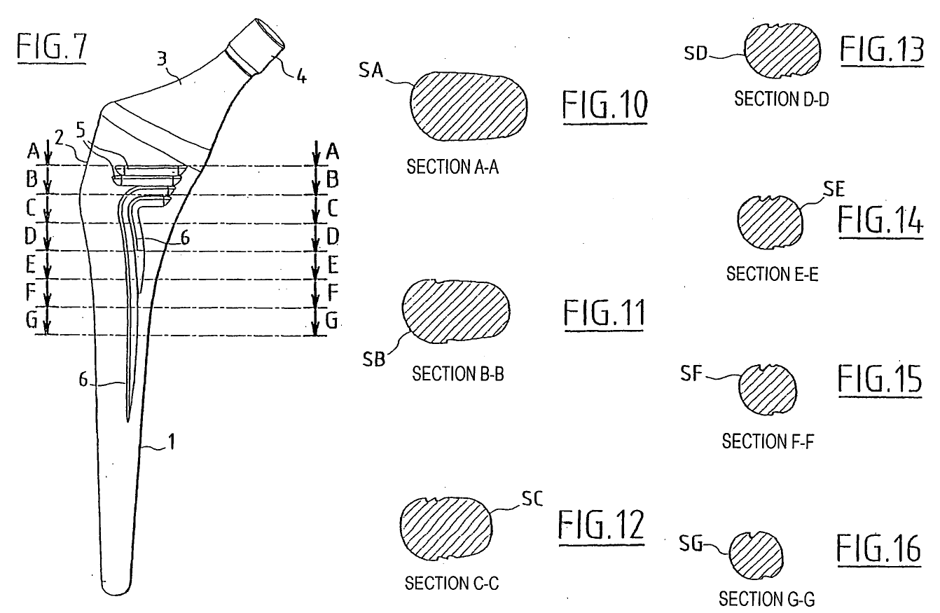

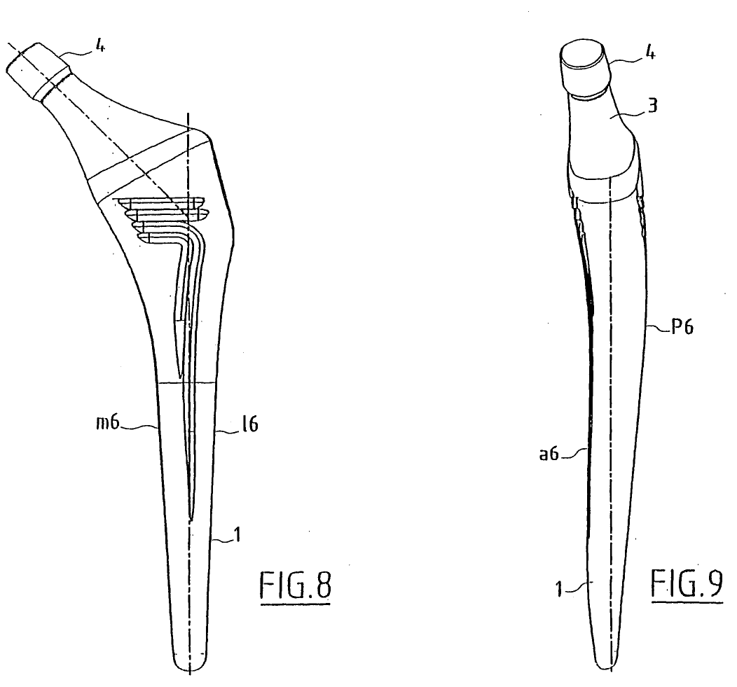

FIGS. 7 and 8 are respectively an anterior view and a posterior view of a prosthesis for the intermediate size, known as size T4.5 for a right leg prosthesis, the plane of the figure being a frontal plane.

FIG. 9 is a view of the median side in the sagittal direction.

FIGS. 10 to 16 are horizontal cross-sections of the prosthesis in planes A to G of FIG. 7.

With reference initially to FIGS. 1 and 2, which relate to a given size of prosthesis and thus to a corresponding size of femur, there are shown the lines that result from projecting the inside wall of the channel of the femur, these projections forming medial and lateral lines m and l in the frontal plane and anterior and posterior lines a and p in the sagittal plane. These lines can be determined by averaging projections onto the frontal and sagittal planes respectively of a significant number of femur cavities corresponding to a determined size of femur. This could be done, for example, using X-rays on a statistically significant population. The metaphyseal portion has been subdivided on seven horizontal planes A, B, C, D, E, F, and G that are equidistant from one another. The intersection between the top plane A and the medial director line m, i.e. the point i, corresponds substantially to the bottom point of the plane where the neck of the femur has been resected by the surgeon while placing a hip prosthesis in a patient of femur size corresponding substantially to that shown in the figures. There is also shown a straight line I defined arbitrarily substantially in a middle zone of the femur shaft and that intersects the various sections A to G at respective points referred to as the “poles” of the sections.

It should also be understood that the director lines m, l, a, and p for other sizes of femur are not geometrically similar to the lines shown in FIGS. 1 and 2, but must be defined using a statistically significant population for each other size of femur.

Ten sets of these four lines appear in FIGS. 5 and 6 for ten different sizes of femur, namely T1, T2, T3, T3.5, T4, T4.5, T5, T5.5, T6, and T7.

With reference to FIG. 3, there is shown a particular section plane, e.g. the plane of proximal section A on which there can be seen traces marking the frontal plane PF and the sagittal plane PS that are drawn to intersect on the arbitrary axis I. In this section plane of FIG. 3, references m, l, a, and p represent the projections of the director lines onto the frontal and sagittal planes PF and PS. Thus, a rectangle RA is defined whose sides are parallel to the planes PF and PS and which pass through projection points m, l, a, and p. Nevertheless, it is possible to use a different quadrilateral, e.g. a rectangle having sides slightly inclined relative to the plane PS or the plane PF, or indeed a trapezoid or some similar quadrilateral.

Finally, there is shown a curve representing a section SA of a prosthesis, its shape being ovoid with two major sides that are substantially flat and that are inscribed inside the rectangle RA which is tangential to the sides, the points of tangency preferably being defined in the plane of the horizontal section for a significant population of femur shafts, with a section of this appearance being quite close to the sections normally used at the level of this plane in known prostheses. It is preferred that the section be fairly flat rather than completely flat on its major sides, and CA and DA are chords subtended from the major arcs of the sections seen from the anterior side and the posterior side.

Between them, the chords CA and DA form a small angle that is constant in all of the sections. This angle preferably depends on the size of the prosthesis, for example in compliance with the following table:

Size: T1, T2, T3, T3.5, T4, T4.5, T5, T5.5, T6, T7.

Angle: 6°20′, 7°20′, 7°37′, 7°53′, 8°11′, 8°14′, 8°19′, 8°24′, 8°31′, 8°40′.

The two minor sides are more rounded, the chords subtended from the small arcs on the minor sides of the rectangle being fairly short.

Reference is now made to FIG. 4.

This figure is a plan view showing seven ovoid curves of sections SA to SG in the various section planes A to G. The geometrical construction is performed as follows:

For example for section plane B, a rectangle RB is drawn of sides that are respectively parallel to the sides of the rectangle RA, the sides of the rectangle RB passing respectively through the points of intersection between the profile lines m, l, a, and p and the plane of section B. Then, in rectangle RB, a curve is drawn of the prosthesis section SB which is inscribed tangentially in the rectangle RB and of orientation such that the chords CB and DB subtended by the two relatively flat major sides form a helitorsion angle relative to the chords CA and DA, which angle corresponds to the desired amount of helitorsion variation between the planes A and B. For example, the preferred helitorsion angle between the chords CA and CB and also between the chords DA and DB is 2°30′, the chords of the two minor faces preferably being offset by the same helitorsion variation on going from one section to another. Naturally, the general appearance of the curve SB is the same as that of the curve SA with the two constraints of being inscribed in the rectangle RB and of having the chords of its major faces offset by the above-mentioned angle relative to the chords of the adjacent section plane A. This construction is continued from section to consecutive section, thereby enabling the various section curves SA to SG to be obtained as shown in FIG. 4. If the seven section planes A to G determining six slices of the prosthesis are taken into consideration when the total helitorsion is 8°, which is the preferred value, then the helitorsion differences going from one plane to the next starting from the plane G and going up towards the plane A are as follows: 30′, 30′, 1°, 1°30′, 2°, 2°30′. Advantageously, the radii of curvature of the arcs of the four corners of the section vary, decreasing on going away from the proximal section A towards the distal section G, so as to obtain a harmonious progression of sections.

It is thus possible to obtain a complex shape for the metaphyseal portion of the prosthesis which can be constructed simply by adopting director lines m, l, a, and p and by subdividing the metaphyseal zone into a plurality of sections, e.g. seven sections, where this number of sections could nevertheless be slightly greater or slightly smaller, with the construction of this extremely complex shape that is particularly well adapted nevertheless being very simple to perform. It can even be performed by manual drawing, by manually determining the various rectangles RA to RG by drawing in one of the planes a prosthesis section curve that is tangential to and inscribed in its rectangle, by optimizing said curve empirically, as is conventional, by determining the chords of the major faces, and by deducing the other curves of the section in compliance with the above-mentioned conditions.

The person skilled in the art can naturally develop in simple manner algorithms that perform these operations or that comply with such determinations as performed manually, said algorithms then being suitable for controlling machine tools for making prostheses.

With reference to FIGS. 5 and 6, there are shown projections onto the frontal and sagittal planes respectively of ten prostheses of different sizes forming a set. It will be understood that for a prosthesis of given size, e.g. the largest prosthesis, and given that the way the prosthesis is constructed in the manner defined above, the outline corresponds to the construction lines m, l, a, and p for this size of prosthesis. For a prosthesis of size 10, the above-mentioned lines are written m₁₀ , l₁₀, a₁₀ and p₁₀. The corresponding curves are also labeled for size 1.

Naturally, the length of the prosthesis shank, the height of the metaphyseal portion, the length of the neck, and possibly also the size of the frustoconical head all vary as is known to the person skilled in the art of this kind of prosthesis.

Reference is now made to FIGS. 7 to 16.

The prosthesis shown is a prosthesis of size T4.5. This prosthesis possesses the following dimensions:

The distance between the section planes A to G is 51.6 mm.

The distance between two adjacent planes is thus 8.6 mm.

The distance between plane A and the bottom plane H from which the rounded terminal portion of the prosthesis shank begins is 126.5 mm.

The sections SA to SG in FIGS. 10 to 16 comply with the rules as defined above. In conventional manner, the prosthesis has a prosthesis shank 1, a metaphyseal portion 2, a neck 3, e.g. a flared neck, and a frustoconical head 4 for receiving a femur ball.

The prosthesis may advantageously have a certain number of grooves. These grooves may comprise a plurality of horizontal grooves 5, the bottom grooves possibly being continued, after a continuously curved portion, by vertical grooves 6. The traces of these grooves can be seen in FIGS. 11 to 16. These vertical grooves 6 are advantageously curved and shaped to follow very exactly the direction followed by the prosthesis as it is lowered into the femur shaft at the moment when the prosthesis has been inserted far enough to make significant contact with the inside surface of the shaft which has previously been treated with a corresponding rasp, thereby implementing a kind of terminal guidance for the prosthesis at the end of its downward movement, and during impacting.

1-16. (canceled)

17. A prosthesis femoral part of the type comprising a prosthesis shank, a prosthesis metaphyseal portion for forming a bearing zone in the metaphysis of the femur, and a head having a suitable anteversion angle, said metaphyseal portion being shaped to present helitorsion, wherein the metaphyseal portion presents a succession of geometrical sections at equidistant levels parallel to a plane that is transverse relative to the general direction of the prosthesis, which sections are in the form of quadrilaterals with greatly rounded corners with two major sides that converge in the medial direction, each section being inscribed in, and tangential on all four sides to, a geometrical quadrilateral situated in the plane of said section at the level in question, which quadrilateral intercepts via its four sides the four respective geometrical lines obtained by projecting on two inclined planes the profiles of the inside surface of the bone cortex of the femur, said sections being turned relative to one another in an inwardly-turning angular direction on passing upwards from one equidistant plane to a consecutive equidistant plane, said turns together leading to the desired helitorsion angle.

18. A prosthesis according to claim 17, wherein said quadrilaterals are rectangles.

19. A prosthesis according to claim 17, wherein said lines are projection profiles onto the frontal and sagittal planes respectively, thereby forming medial, lateral, anterior, and posterior profiles.

20. A prosthesis according to claim 17, wherein the total helitorsion angle between the extreme planes is 6° to 12°, and preferably 8° to 10°.

21. A prosthesis according to claim 17, wherein the variations in helitorsion between two consecutive planes, when subdivided into seven planes, lies in the range 15′ to 3°.

22. A prosthesis according to claim 20, wherein the helitorsion variation is such that when subdivided into seven consecutive equidistant planes, and for a total helitorsion of 8°, the successive helitorsion angles are 30′, 30′, 1°, 1°30′, 2°, 2°30′ in the proximal direction, each of said values possibly varying by ±30′.

23. A prosthesis according to claim 17, wherein the profiles are, as a function of prosthesis size, those shown in FIGS. 5 and 6.

24. A prosthesis according to claim 17, wherein the convergence angle between the major sides of each geometrical section is of the order of 5° to 10°.

25. A prosthesis according to claim 17, wherein the major sides of the rounded quadrilateral section presents curved major sides with a large angle of curvature.

26. A prosthesis according to claim 25, wherein the large radii of curvature remain substantially constant in all of the sections.

27. A prosthesis according to claim 17, including grooves in its metaphyseal and/or diaphyseal portion, the grooves serving to improve anchoring and osteo-integration, the grooves being horizontal in the upper metaphyseal portion and longitudinal in the lower metaphyseal portion and in the following prosthesis shank.

28. A prosthesis according to claim 27, wherein the longitudinal groove(s) follow the anatomical shape of the prosthesis shank in the lower metaphyseal portion and the diaphyseal portion in such a manner that during insertion of the prosthesis into the femur shaft, the groove produces a guidance effect without forcing on the bone matter.

29. A set of hip prosthesis femoral parts according to claim 17, wherein they comply with a set of profile lines corresponding to their sizes, as defined in FIGS. 5 and 6 for a set of ten different sizes.

30. A method of manufacturing a prosthesis according to claim 17, wherein for a given size of prosthesis, four projection profiles are determined by projecting the corresponding inside cortex surfaces, e.g. by X-ray or by scanner, in particular onto two perpendicular planes, in particular the sagittal and frontal planes giving lateral, medial, anterior, and posterior profiles, wherein a quadrilateral, in particular a rectangle is determined in said plane which is determined as one of a succession of equidistant parallel cross-section planes spaced apart along the metaphyseal portion of the prosthesis with the bottom plane forming the distal metaphyseal section, wherein, in each of these planes, the quadrilaterals are defined of sides that remain respectively parallel, said quadrilaterals intersecting the various profile curves with their different sides in the plane at the level under consideration, and wherein there is drawn in each of the quadrilaterals a section of the metaphyseal portion which is tangential to the sides of the quadrilateral in question, having major sides that are inclined relative to the major sides of the section immediately above by a given helitorsion angle, the sum of said helitorsion angles from the proximal section to the distal section determining the total helitorsion angle, wherein the data set determined in this way is stored, and wherein a femur head manufacturing device is controlled thereby, said device being of any type, e.g. operating by molding, machining, or forging.

31. A method of manufacturing a set of prostheses, implementing the method according to claim 30 for each individual prosthesis, all of the prostheses in the set being determined by said method as a function of the size determined by the profiles.

32. A method according to claim 30, wherein a geometrical axis is defined forming a point in the proximal plane, said geometrical axis being, in particular, substantially a mean diaphyseal axis, with the sides of the proximal section rectangle being identified under such circumstances by values Xₘₗ and Yₐₚ, and forming the portions of the major side and of the minor side respectively of the rectangle to have values Xₘₗ=45% ml, Yₐₚ=28% ml.
